(19) United States
(12) Patent Application Publication
(10) Pub. No.: US 2007/0162117 A1
(43) Pub. Date: Jul. 12, 2007

(54) Iris prothesis system

(76) Inventor: Heino Hermeking, Wuppertal (DE)

Correspondence Address: Thomas M Galgano;Galgano & Burke, 300 Rabro Drive, Suite 135204, Hauppauge, NY, 11788, US

(21) Appl. No.: 10/555,918

(22) PCT Filed: May 6, 2004
(86) PCT No.: PCT/DE04/00961

§ 371 Date: Oct. 5, 2006

(30) Foreign Application Priority Data
May 7, 2003 (DE) 103 20 584.5

(51) Int. Cl. A61F 2/16

(52) U.S. Cl. 623/6.17; 623/6.41; 623/4.1

(57) ABSTRACT

Diaphragms that can be implanted in the anterior section of a human eye, individually or combined with one another, in order to create an artificial pupil opening, are part of a modular iris prosthesis system. At least one diaphragm that can be implanted in endocapsulary manner, at least one diaphragm that can be implanted in sulcus-positioned manner, and at least one diaphragm that can be implanted in iris-enclaved and/or sclera-fixed manner are provided.

It is the task of the invention to create a modular iris prosthesis system, which makes it possible to create an artificial pupil opening under the most varied medical starting conditions.

The iris prosthesis system that accomplishes this task includes diaphragms that can be implanted into the anterior section of a human eye, individually or combined with one another, in order to create an artificial pupil opening. The modular system includes at least one diaphragm that can be implanted in endocapsulary manner, at least one diaphragm that can be implanted in sulcus-positioned manner, and at least one diaphragm that can be implanted in iris-enclaved and/or sclera-fixed manner.

Diaphragms of an iris prosthesis system that can be implanted in endocapsulary manner are known from WO 98/56 314 A1.

In the case of one preferred embodiment, a diaphragm that can be implanted in sulcus-positioned manner is provided, which has a coverage region on one side, along with a central aperture and a stirrup-shaped haptic on the other side of it.

In the case of one preferred embodiment, the haptic is C-shaped.

In the case of one preferred embodiment, the diaphragm is provided with at least one opening for a needle tip or a guide hook.

In the case of one preferred embodiment, a diaphragm that can be implanted in sulcus-positioned or iris-enclaved and/or sclera-fixed manner consists of essentially rigid planar elements that are divided on at least one folding line. The division is bridged with an elastic material that enters into an adhesive connection with the planar elements, that allows the diaphragm to be folded in half elastically, so that the latter is suitable for unfolding back into its original position by means of its inherent elasticity.

A preferred embodiment of the diaphragm has at least one straight fold line.

Preferred embodiments of the diaphragm have either a single or two or more parallel fold lines.

In the case of one preferred embodiment, the elastic material that bridges the division of the diaphragm is silicone (polyorganosiloxane)

In the case of one preferred embodiment, the planar elements of the diaphragm are provided with holes close to the edge, on both sides of the division. These make it possible for the elastic material to penetrate the planar elements, passing through them completely, and to enter into a particularly intimate adhesive bond with the planar elements. The holes are preferably round holes.

In the case of one preferred embodiment, a foldable fixation ring is provided, which can be inserted into a central aperture of the diaphragms and reinforces them. A suitable fixation ring is the object of DE 101 56 463 A1.

In the case of one preferred embodiment, a lens is provided, which can be clipped into a central aperture of the diaphragms or of the fixation ring.

In the case of one preferred embodiment, the lens has symmetry of rotation relative to a central axis crosswise to its main plane. It has a lens body that is curved convex towards the outside, behind that a diameter setback, and adjacent to that an anchoring part that is curved convex towards the outside, having a greater diameter.

In the case of one preferred embodiment, the diameter of the lens body is greater than that of the anchoring part.

In the case of one preferred embodiment, the diaphragms consist of polymethyl methacrylate (PMMA) that is preferably dyed, particularly dyed with pigment dye. The lens consists of hydrophilic acrylate.

The invention will be explained in greater detail in the following, using exemplary embodiments shown in the drawing. This shows:

FIG. 1 a top view of a single diaphragm that can be implanted in endocapsulary manner;

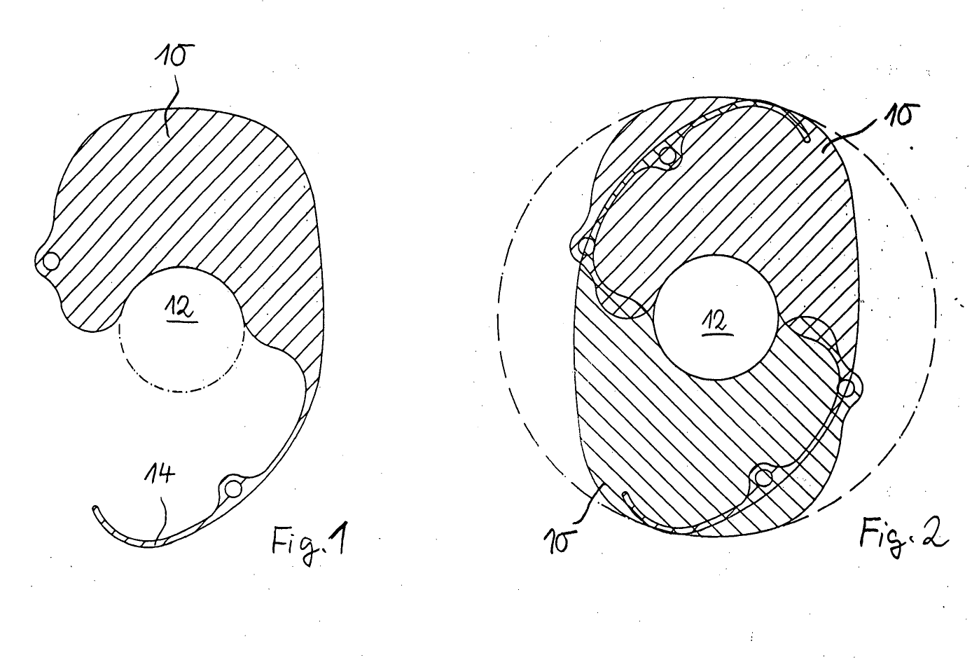

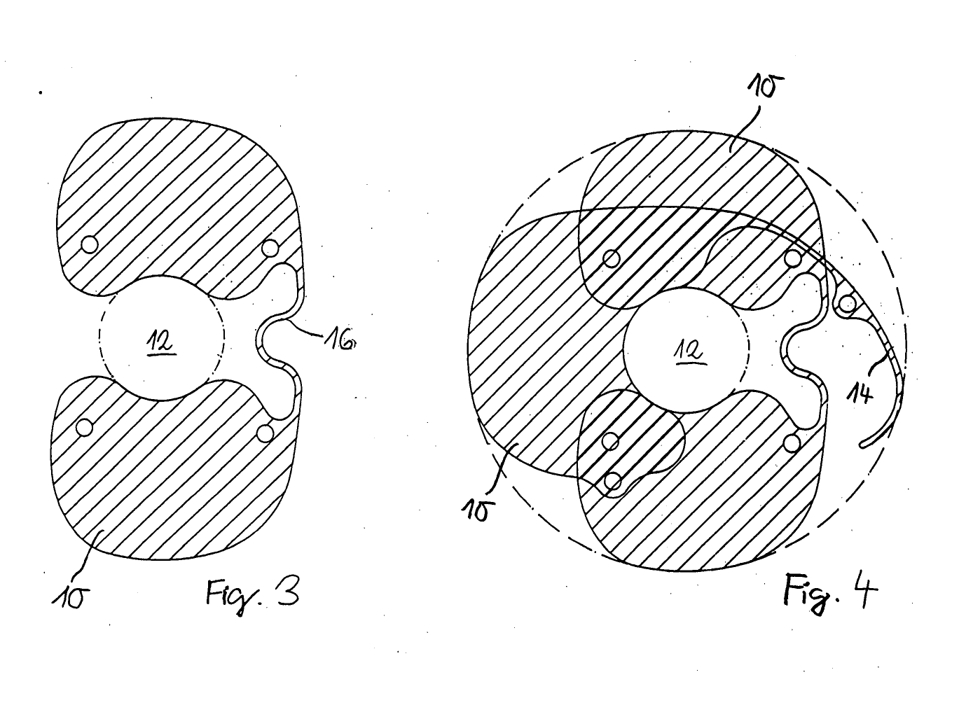

FIG. 2 a top view of two single diaphragms according to FIG. 1, which are in contact with one another offset by 180°;

FIG. 3 a top view of a double diaphragm that can be implanted in endocapsulary manner;

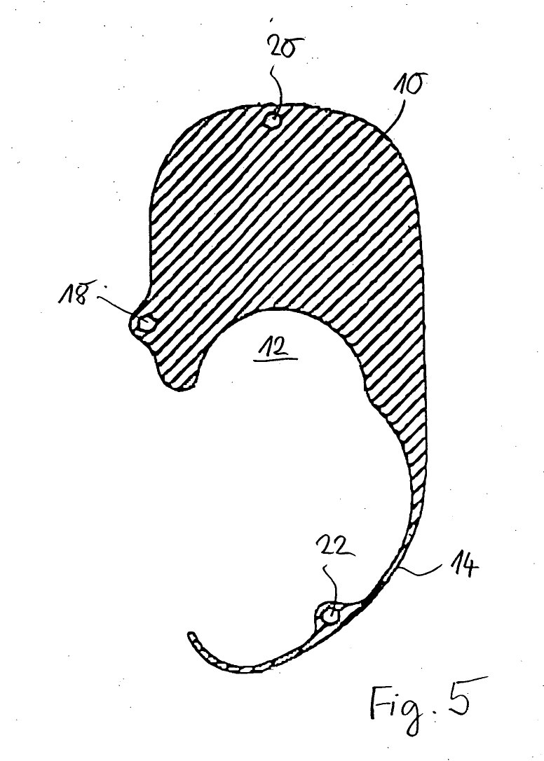

FIG. 4 a top view of a single diaphragm according to FIG. 1 and a double diaphragm according to FIG. 3, which are in contact with one another offset by 90°;

FIG. 5 a top view of a single diaphragm that can be implanted in sulcus-positioned manner;

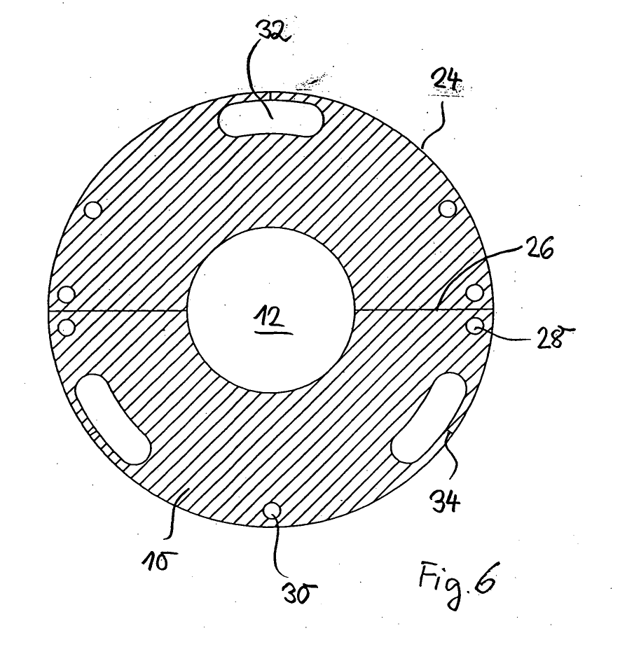

FIG. 6 a top view of a foldable diaphragm that can be implanted in iris-enclaved and/or sclera-fixed manner;

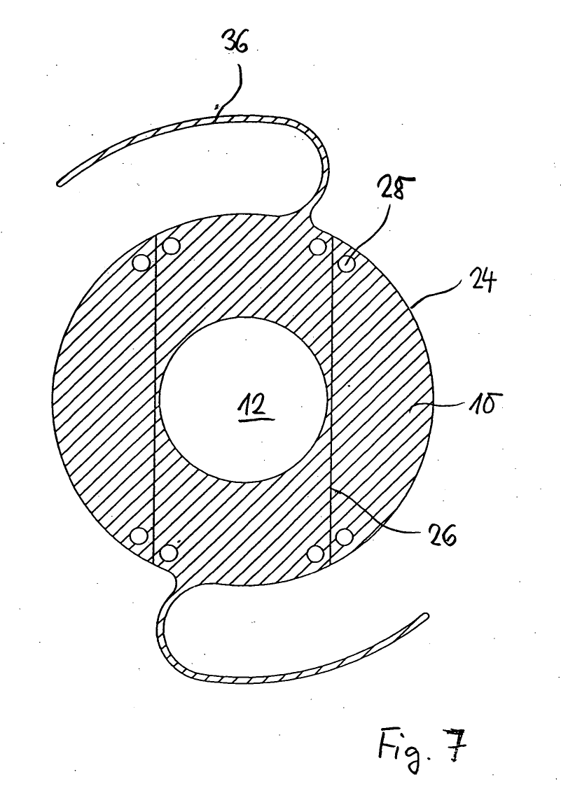

FIG. 7 a top view of a foldable diaphragm that can be implanted in sulcus-positioned manner;

FIG. 8 a top view of a fixation ring;

FIG. 9 a side view of the fixation ring with a view in the direction IX of FIG. 8;

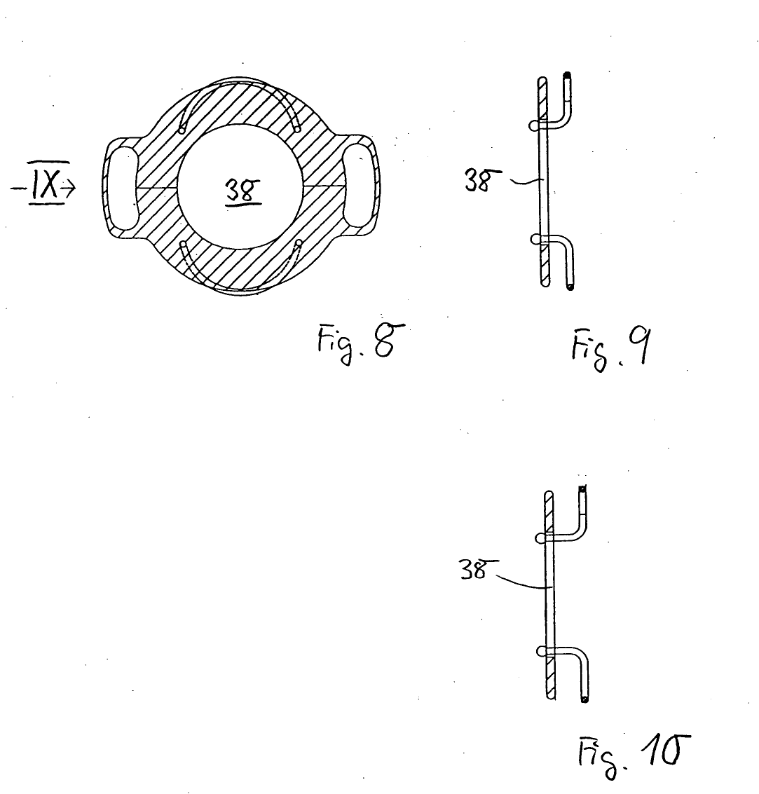

FIG. 10 a side view of the fixation ring in an alternative embodiment;

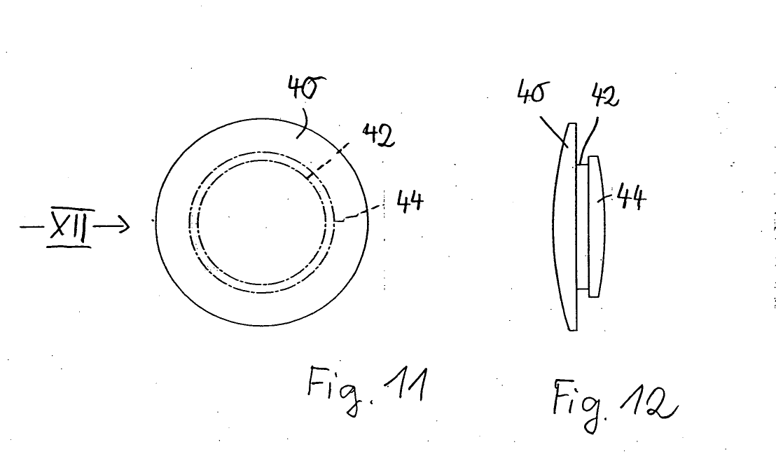

FIG. 11 a top view of a lens; and

FIG. 12 a side view of the lens with a view in the direction XII of FIG. 11.

The diaphragms are planar parts made of a polymethyl methacrylate dyed with pigment dye, in the colors brown, green, blue. This material is characterized by good, assured compatibility (biocompatibility), while greatly limiting light transmission.

The single diaphragm that can be implanted in endocapsulary manner, shown in FIG. 1 and FIG. 2, has a coverage region 10 on one side next to a central aperture 12. The single diaphragm has a C-shaped haptic 14.

In the case of implantation together with an artificial lens in the lens capsule bag, the single diaphragm by itself serves to cover iris colobomas (iris gaps).

As shown in FIG. 2, in the case of double implantation of the diaphragm in the capsule bag, formation of a pupil is achieved. The two single diaphragms are in flush contact with one another, offset by 180°.

The double diaphragm that can be implanted in endocapsulary manner, shown in FIG. 3, has two coverage regions 10 that have the same shape and lie diametrically opposite one another and extend on both sides next to a central aperture 12. The coverage regions 10 are connected, on one side of the aperture, by means of an elastic stirrup 16 that is curved in U shape towards the inside and leaves the aperture 12 open.

During implantation into the lens capsule bag, together with an artificial lens, the double diaphragm by itself serves to cover opposite iris defects having a small expanse. An example is iatrogenic sphincterotomies that were placed to treat narrow pupils, before performing a cataract operation.

An approximately circular artificial pupil opening can be created with two double ends that lie one behind the other and are offset relative to one another by 90°. Also, as shown in FIG. 4, single and double diaphragms are compatible with one another for common implantation into the lens capsule bag.

FIG. 5 shows a single diaphragm that can be implanted in sulcus-positioned manner, having a coverage region 10 on one side, next to a central aperture 12 and a C-shaped haptic 14. The shape of the diaphragm is similar to that of the single diaphragm that can be implanted in endocapsulary manner. Like the latter, the diaphragm can also be implanted in pairs with a 180° offset, in order to create an artificial pupil opening. However, the sulcus-positioned diaphragm has a significantly greater diameter than the endocapsulary diaphragm.

The haptic 14 begins on the coverage region 10 of the sulcus-positioned diaphragm, on one side. It narrows towards its end, and leaves the central aperture 12 free.

At the edge of the coverage region 10, two round openings 18, 20 for a guide hook are provided. Another such opening 22 is situated on the haptic 14, close to its end. The diaphragm does not have a fold line.

FIG. 6 shows a diaphragm that can be implanted in iris-enclaved and/or sclera-fixed manner. The diaphragm has the basic shape of a flat circular ring having a central circular aperture 12, and an outer circumference circle 24 that runs concentric to the edge of the aperture.

The diaphragm is divided by means of a diametrical fold line 26. The halves are connected at the fold line with an elastic adhesive seam of silicone 26.

The diaphragm is provided with round holes 28 disposed close to its outer edge, in a mirror symmetry arrangement, on both sides of the fold line 26. These are four round holes 28 having the same size, which lie opposite one another in pairs with reference to the fold line 26. Thanks to the round holes 28, the elastic material of the adhesive seam can penetrate the halves.

The diaphragm has three additional round holes 30 having the same size, which lie close to the edge and lie on the same circumferential circle as the round holes 28, on both sides of the fold line 26, offset by 120° from one another. The round holes 30 serve for sclera fixation of the diaphragm with a suture.

Centered between the round holes 30, the diaphragm is provided with significantly larger, essentially oval elongated holes 32 that lie close to the edge. Their long edges are rounded, essentially following the outer periphery of the diaphragm. A central, radial incision 34 extends from the outer long edge to the outer circumference circle 24. The incisions 34 allow enclavation of iris tissue. One of the round holes 30 and elongated holes 32 is offset from the fold line 26 by 90°, in each instance.

FIG. 7 shows a diaphragm that can also be folded, which can be implanted in sulcus-positioned manner. The diaphragm has a circular, planar central body having a central, circular aperture 12, and an outer circumference circle 24 that runs concentric to the edge of the aperture. Two haptic stirrups 36 that lie opposite one another start on the outside of the central body; initially, they are curved forward in the clockwise direction, and during their further progression, they are curved backward parallel to the outer circumference circle 24 of the central body.

The central body of the diaphragm is divided twice, essentially crosswise to the haptic stirrups 36, with two straight fold lines 26. The fold lines 26 extend parallel, at a slight distance from one another, past the aperture 12, on both sides. The segments of the central body are connected at the fold lines 26 with elastic adhesive seams made of silicone.

The diaphragm is provided with round holes 28 that are disposed close to its outer edge, on both sides of the fold lines 26. These are four pairs of round holes 28 that lie on the same circumference circle and are spaced at the same distance from the fold lines 26, in pairs. Thanks to the round holes 28, the elastic material of the adhesive seam can penetrate the segments of the diaphragm.

The diaphragm according to FIG. 7 has dual symmetry of rotation. It makes a transition into itself when turned by 180° about its center point.

The fixation ring according to FIG. 8 to FIG. 10 is the object of DE 101 56 463 A1. It can be inserted into the central aperture 12 of all the diaphragms of the iris prosthesis system, in order to stabilize them. The fixation ring in turn has a central aperture 38.

FIG. 11 and 12 show a lens made of hydrophilic acrylate. The lens can be clipped either into the central aperture 12 of the diaphragms, or into the central aperture 38 of the fixation ring, depending on its size. The lens has symmetry of rotation relative to a central axis crosswise to its main plane. It has a lens body 40 that is curved convex towards the outside, with a round cylindrical diameter setback 42 behind that. An anchoring part 44 that is also curved convex towards the outside, having a greater diameter, follows adjacent to the latter. The diameter of the lens body 40 is greater than that of the anchoring part 44. After the lens is clipped in, the diameter setback 42 determines the optically exposed lens part.

The diaphragms according to FIG. 5 to FIG. 7 are created for anatomically pathological situations in which the aim is to produce a diaphragm ring in the anterior section of the eye, to compensate for iris tissue, specifically in those cases where prosthetic positioning in the capsule bag is no longer possible. In this connection, the diaphragmal iris prosthesis system can be sulcus-positioned. Here, although the capsule bag is still present, endocapsulary provision of the prosthesis is no longer possible for various reasons. The capsule merely serves as a support for positioning of the prosthesis in the sulcus ciliaris.

The diaphragms according to FIG. 5 and FIG. 7 are designed for this location of use. Fundamentally, the diaphragm according to FIG. 6 can also be positioned in this region. However, a prerequisite for fixation of this diaphragm is a residual amount of iris tissue that can allow retroiridal fixation of the prosthesis, for example. Alternatively, the prosthesis is fixed in place by means of a sclera suture. In these cases, the presence of a capsulary support is not necessary, because of the two fixation possibilities just mentioned.

The diaphragm according to FIG. 6 can also be mounted in preiridal manner. In this connection, it is necessary for a residual amount of iris tissue to be present for fixation of the diaphragmal disk, if the disk is supposed to be fixed in preiridal manner. Suture fixation of the disk in the preiridal position is prohibited.

The invention allows intraocular implantation of diaphragmal diaphragm disks into the anterior eye section, using the technique of small incision surgery, especially if there is no possibility of positioning the prosthesis in endocapsulary manner.

The diaphragmal disks can be folded along fold lines 26 that divide the prosthesis into two or three parts. Cohesion of the prosthesis is guaranteed in that the fold lines 26 are bridged by a silicone layer or adhesive seam. The adhesive consists of the silicone. In the unfolded state, the outside diameter of the diaphragm according to FIG. 6 is approximately 10.5 mm, and in the case of the diaphragm according to FIG. 7, the outside diameter of the central body is approximately 9 mm. In the folded state, the diaphragm according to FIG. 6 can be reduced to a diameter of approximately 5.25 mm, whereby the folded faces have a width of only approximately 3.25 mm, in each instance. This width is the determining factor for the incision size, taking the thickness of the prosthesis (approximately 0.2 mm) into consideration at the same time. In the case of the diaphragm according to FIG. 7, which has two fold lines 26, the width of the central body is reduced from approximately 9 mm to approximately 4.4 mm in the folded state. Taking the prosthesis thickness (approximately 0.2 mm) into consideration added to that, this dimension also determines the required incision size.

The diaphragm according to FIG. 6, for iris enclavation and sclera fixation, is a circular disk having an outside diameter of approximately 10.5 mm. The diaphragm has three elongated holes 32 for iris enclavation. A claw device makes it possible to draw iris tissue into these elongated holes 32 and fix it there. Furthermore, the diaphragmal disk of the diaphragm has round holes 30 that serve to accommodate sutures during sclera fixation. The aperture 12 of the diaphragmal disk has a diameter of approximately 4 mm. The unfolded state is stabilized by means of the insertion of the fixation ring, which is also foldable, shown in FIG. 8 to FIG. 10, which ring is inserted into the diaphragm with its fold line extending perpendicular to the fold line 26 of the diaphragm. This ensures that the diaphragmal disk remains in the unfolded state and is stabilized there. The diameter of the aperture 38 of the fixation ring is approximately 3 mm. There is the possibility of clipping the lens according to FIG. 11 and FIG. 12 into this aperture 38. The lens can be configured according to the individual optical requirements.

The prosthesis according to FIG. 7, for sulcus positioning, has a diaphragmal disk having a diameter of approximately 9.0 mm. To reduce the size during implantation, the disk has two fold lines 26. The fold lines are provided with two round holes 28 at their ends, in each instance. The central part of the diaphragmal prosthesis carries a haptic 36 on the outside, in each instance, the outside diameter of which is approximately 13.75 mm. The haptic 36 serves for positioning in the sulcus ciliaris. The central aperture 12 of the diaphragm according to FIG. 7 has a diameter of approximately 4 mm. The aperture 12 can also serve for accommodating the aforementioned fixation ring or a lens that can be clipped into it. The front lens body 40 of the lens has a diameter of approximately 5 mm, the diameter setback 42 has a diameter of approximately 3 mm, and the rear anchoring part 44 has a diameter of approximately 3.4 mm.

### LIST OF REFERENCE SYMBOLS

10 coverage region
12 aperture of the diaphragm
14 haptic
16 stirrup
18 opening for guide hook
20 opening for guide hook
22 opening for guide hook
24 circumference circle
26 fold line
28 round hole for adhesive seam
30 round hole for sclera fixation
32 elongated hole
34 incision
36 haptic stirrup
38 aperture of the fixation ring
30 lens body
42 diameter setback
44 anchoring part

1. Iris prosthesis system with diaphragms that can be implanted in the anterior section of a human eye, individually or combined with one another, in order to create an artificial pupil opening, having at least one diaphragm that can be implanted in endocapsulary manner, at least one diaphragm that can be implanted in sulcus-positioned manner, and at least one diaphragm that can be implanted in iris-enclaved and/or sclera fixed manner.

2. Iris prosthesis system according to claim 1, wherein the diaphragm that can be implanted in sulcus-positioned manner has a coverage region on one side, along with a central aperture and a stirrup-shaped haptic on the other side of it.

3. Iris prosthesis system according to claim 2, wherein the haptic is C-shaped.

4. Iris prosthesis system according to claim 2, wherein the diaphragm is provided with at least one opening for a needle tip or a guide hook.

5. Iris prosthesis system according to claim 1, wherein the diaphragm that can be implanted in sulcus-positioned or iris-enclaved and/or sclera-fixed manner consists of essentially rigid planar elements that are divided on at least one fold line, and that the division is bridged with an elastic material that enters into an adhesive connection with the planar elements, that allows the diaphragm to be folded in half elastically, and that the latter is suitable for unfolding back into its original position by means of its inherent elasticity.

6. Iris prosthesis system according to claim 5, wherein least one straight fold line.

7. Iris prosthesis system according to claim 5, wherein the diaphragm has a single fold line.

8. Iris prosthesis system according to claim 6, wherein the diaphragm has two or more parallel fold lines.

9. Iris prosthesis system according to claim 1, wherein the elastic material is silicone (polyorganosiloxane).

10. Iris prosthesis system according to claim 1, wherein the planar elements of the diaphragm are provided with holes close to the edge, on both sides of the division.

11. Iris prosthesis system according to claim 10, wherein the holes are round holes (28).

12. Iris prosthesis system according to claim 1, wherein a foldable fixation ring that can be inserted into a central aperture of the diaphragms.

13. Iris prosthesis system according to claim 1, wherein a lens that can be clipped into a central aperture of the diaphragms or of the fixation ring.

14. Lens, particularly lens for an iris prosthesis system according to claim 1, which has symmetry of rotation relative to a central axis crosswise to its main plane, a lens body that is curved convex towards the outside, behind that a diameter setback, and adjacent to that an anchoring part that is curved convex towards the outside, having a greater diameter.

15. Lens according to claim 14, wherein the diameter of the lens body is greater than that of the anchoring part.

16. Iris prosthesis system according to claim 1, wherein the diaphragms consist of polymethyl methacrylate (PMMA) that is preferably dyed, particularly dyed with pigment dye, and that the lens consists of hydrophilic acrylate.
